(19) United States
(12) Patent Application Publication
(10) Pub. No.: US 2025/0222405 A1
(43) Pub. Date: Jul. 10, 2025

(54) MEMBRANE SEPARATION AND PURIFICATION APPARATUS, AND METHOD THEREOF

(71) Applicant: ZHEJIANG HYMATER NEW MATERIALS CO., LTD, Ningbo, Zhejiang (CN)

(72) Inventor: Yanshuo LI, Ningbo, Zhejiang (CN)

(21) Appl. No.: 18/853,871

(22) PCT Filed: Apr. 6, 2023
(86) PCT No.: PCT/CN2023/086468

§ 371 Date: Oct. 3, 2024

(30) Foreign Application Priority Data
Apr. 8, 2022 (CN) 202210368852.0
Apr. 8, 2022 (CN) 202220815738.3

(51) Int. Cl. B01D 61/36; B01D 3/00; B01D 3/14; B01D 3/36; B01D 15/10

(52) U.S. Cl. CPC B01D 61/368; B01D 3/007; B01D 3/145; B01D 3/36; B01D 15/10; B01D 61/366

(57) ABSTRACT

A membrane separation and purification apparatus includes a heating module, N levels of a membrane separation module, N first transmission pipelines, and at least N−1 second transmission pipelines, with N being a an integer not less than 2. Each level of the membrane separation module is used to perform a membrane separation and purification treatment on a material, the heating module heats the first level of the membrane separation module, adjacent two levels of the membrane separation module are connected via one of the first transmission pipelines and one of the second transmission pipelines, the first transmission pipeline introducing the product from a preceding level of the membrane separation module into a subsequent level of the membrane separation module for a first heat exchange, the second transmission pipeline outputting the product after the first heat exchange into the preceding level of the membrane separation module for a second heat exchange.

### TECHNICAL FIELD

The present application relates to the technical field of organic azeotropic purification, specifically to a membrane separation and purification apparatus and a membrane separation and purification method.

### BACKGROUND ART

In the chemical industry, it is often necessary to separate and purify organic raw materials such as alcohols, ketones, aldehydes, ethers, and esters, in order to remove impurities like water from chemical raw materials and obtain products of higher purity. However, since the aforementioned organic raw materials and water form an azeotropic system, it is challenging to separate them using conventional separation methods, making it difficult to obtain high-purity products. To achieve separation, methods such as azeotropic distillation method, extractive distillation method, adsorption separation method are commonly used. However, these methods currently face problems such as low heat utilization efficiency, high energy consumption, and non-compliant direct discharge of wastewater.

### SUMMARY OF INVENTION

Embodiments of the present application provide a membrane separation and purification apparatus and a membrane separation and purification method to solve the problems, such as low heat utilization efficiency and high energy consumption, during the separation and purification of chemical raw materials in existing azeotropic systems.

To achieve the above invention objectives, the technical solution of the present application is as follows.

A membrane separation and purification apparatus includes: a heating module; N levels of a membrane separation module; N first transmission pipelines; and at least N−1 second transmission pipelines, with N being a positive integer not less than 2, wherein
each level of the membrane separation module is used to perform a membrane separation and purification treatment on a material to obtain a product correspondingly,
a first level of the membrane separation module is heated by the heating module to perform the membrane separation and purification treatment,
adjacent two levels of the membrane separation module are connected respectively via one of the first transmission pipelines and one of the second transmission pipelines, the first transmission pipeline introducing the product generated by a preceding level of the membrane separation module into a subsequent level of the membrane separation module to perform a first heat exchange with a material introduced into the subsequent level of the membrane separation module, the second transmission pipeline outputting the product that has undergone the first heat exchange into the preceding level of the membrane separation module to perform a second heat exchange with a material introduced into the preceding level of the membrane separation module, and
an Nth level of the membrane separation module introduces the product generated by the Nth level of the membrane separation module into the Nth level of the membrane separation module via one of the first transmission pipelines to perform a third heat exchange with a material introduced into the Nth level of the membrane separation module.

In some embodiments, each level of the membrane separation module includes a first heat exchanger, a third transmission pipeline, a first evaporator, a fourth transmission pipeline, and at least one membrane component, which are connected sequentially,
the heating module provides heat to the first evaporator in the first level of the membrane separation module, and
between the adjacent two levels of the membrane separation module, a feed end of the first transmission pipeline connects to the membrane component in the preceding level of the membrane separation module, and a discharge end of the first transmission pipeline connects to the first evaporator of the subsequent level of the membrane separation module, a feed end of the second transmission pipeline connects to the first evaporator of the subsequent level of the membrane separation module, and a discharge end of the second transmission pipeline connects to the first heat exchanger of a current level of the membrane separation module.

In some embodiments, each level of the membrane separation module further includes a fifth transmission pipeline, and each level of the membrane separation module includes a first membrane component and a second membrane component,
the fourth transmission pipeline connects the first evaporator and the first membrane component, the fifth transmission pipeline connects the first membrane component and the second membrane component, one end of the first transmission pipeline, which connects the adjacent two levels of the membrane separation module, connects to the second membrane component,
each level of the membrane separation module further includes a permeate condensation component which connects to the membrane component to perform a permeate condensation treatment on a permeate vapor generated by the membrane component,
or each level of the membrane separation module further includes a permeate condensation component, a first membrane component, and a second membrane component, the permeate condensation component in each level of the membrane separation module includes two permeate condensers and two cooling water pipelines, and the membrane separation and purification apparatus further includes a vacuum module and a cold water module, and
the first membrane component connects to one of the permeate condensers, one of the cooling water pipelines introduces water at 5° C. to 15° C. into the permeate condenser connected to the first membrane component, the second membrane component connects to the other permeate condenser, and the other cooling water pipeline introduces water at −15° C. to −5° C. into the permeate condenser connected to the second membrane component.

In some embodiments, a value of the N is 2, 3, or 4,
and/or the membrane separation and purification apparatus further includes a storage module that is used to recover a permeate generated by each level of the membrane separation module.

In some embodiments, the membrane separation and purification apparatus further includes a cooling module that is used to perform a cooling treatment on the product that has undergone the third heat exchange through the Nth level of the membrane separation module,
or the membrane separation and purification apparatus further includes a cooling module that is used to perform a cooling treatment on the product that has undergone the third heat exchange through the Nth level of the membrane separation module, and a circulating water module that connects to the cooling module.

In some embodiments, the first transmission pipeline connected to the Nth level of the membrane separation module includes a first branch and a second branch,
the membrane separation and purification apparatus further includes a functional heat exchange module,
the first branch connects the Nth level of the membrane separation module and the functional heat exchange module to introduce the product generated by the Nth level of the membrane separation module into the functional heat exchange module to perform a fourth heat exchange,
the second branch connects the functional heat exchange module and the Nth level of the membrane separation module to introduce the product that has undergone the fourth heat exchange into the Nth level of the membrane separation module to perform the third heat exchange,
the functional heat exchange module includes any one of a distillation module and a molecular sieve adsorption module,
the distillation module includes a second heat exchanger, a distillation column, a reboiler, a third condenser, a seventh transmission pipeline, an eighth transmission pipeline, a ninth transmission pipeline, a tenth transmission pipeline, and an eleventh transmission pipeline,
a discharge end of the first branch connects to the reboiler, and a feed end of the second branch connects to the reboiler,
the seventh transmission pipeline connects the second heat exchanger and the distillation column to introduce a material that has passed the second heat exchanger into the distillation column,
the eighth transmission pipeline connects the distillation column and the reboiler to introduce a tower bottom liquid generated by the distillation column into the reboiler,
the ninth transmission pipeline connects the reboiler and the distillation column to transfer the tower bottom liquid heated by the reboiler into the distillation column,
the tenth transmission pipeline connects the reboiler and the second heat exchanger to transfer the tower bottom liquid that has been heated by the reboiler into the second heat exchanger to provide heat to the second heat exchanger,
the eleventh transmission pipeline connects the distillation column and the third condenser to transfer a gaseous azeotropic material discharged from a top of the distillation column into the third condenser to perform a condensation treatment,
the membrane separation and purification apparatus further includes a circulating water module that connects to the third condenser,
and/or the membrane separation and purification apparatus further includes a recovery device that connects to the third condenser to collect an azeotrope discharged by the third condenser,
and/or each level of the membrane separation module also connects to the second heat exchanger to introduce the permeate generated by each level of the membrane separation module into the second heat exchanger.

In some embodiments, the molecular sieve adsorption module includes a third heat exchanger, a twelfth transmission pipeline, a second evaporator, a thirteenth transmission pipeline, and at least one adsorption tower component, which are connected sequentially,
the discharge end of the first branch connects to the second evaporator, and the feed end of the second branch connects to the second evaporator, and
the molecular sieve adsorption module further includes a fourteenth transmission pipeline that connects the adsorption tower component and the third heat exchanger to deliver a vapor generated by the adsorption tower component to the third heat exchanger to perform a sixth heat exchange with a material introduced into the third heat exchanger.

A membrane separation and purification method according to the present application includes:
introducing a material into each level of a membrane separation module and heating a first level of the membrane separation module through a heating module to perform a membrane separation and purification treatment; and
controlling adjacent two levels of the membrane separation module so that a flow rate of a material entering a preceding level of the membrane separation module is greater than a flow rate of a material entering a subsequent level of the membrane separation module and a latent heat value of a material entering the preceding level of the membrane separation module is not less than a latent heat value of a material entering the subsequent level of the membrane separation module, wherein
the membrane separation and purification method utilizes a membrane separation and purification apparatus to perform membrane separation and purification,
the membrane separation and purification apparatus includes: the heating module, N levels of the membrane separation module, N first transmission pipelines, and at least N−1 second transmission pipelines, with N being a positive integer not less than 2,
each level of the membrane separation module is used to perform the membrane separation and purification treatment on a material to obtain a product correspondingly,
the first level of the membrane separation module is heated by the heating module to perform the membrane separation and purification treatment,
adjacent two levels of the membrane separation module are connected respectively via one of the first transmission pipelines and one of the second transmission pipelines, the first transmission pipeline introducing a product generated by a preceding level of the membrane separation module into a subsequent level of the membrane separation module to perform a first heat exchange with a material introduced into the subsequent level of the membrane separation module, the second transmission pipeline outputting the product that has undergone the first heat exchange into the preceding level of the membrane separation module to perform a second heat exchange with a material introduced into the preceding level of the membrane separation module,
an Nth level of the membrane separation module introduces a product generated by the Nth level of the membrane separation module into the Nth level of the membrane separation module via one of the first transmission pipelines to perform a third heat exchange with a material introduced into the Nth level of the membrane separation module, and
each of the materials contains an organic azeotrope.

In some embodiments, each level of the membrane separation module includes a first heat exchanger, a third transmission pipeline, a first evaporator, a fourth transmission pipeline, and at least one membrane component, which are connected sequentially,
the heating module provides heat to the first evaporator in the first level of the membrane separation module, and
between the adjacent two levels of the membrane separation module, a feed end of the first transmission pipeline connects to the membrane component in the preceding level of the membrane separation module, and a discharge end of the first transmission pipeline connects to the first evaporator of the subsequent level of the membrane separation module, a feed end of the second transmission pipeline connects to the first evaporator of the subsequent level of the membrane separation module, and a discharge end of the second transmission pipeline connects to the first heat exchanger of a current level of the membrane separation module.

In some embodiments, each level of the membrane separation module further includes a fifth transmission pipeline, and each level of the membrane separation module includes a first membrane component and a second membrane component,
the fourth transmission pipeline connects the first evaporator and the first membrane component, the fifth transmission pipeline connects the first membrane component and the second membrane component, one end of the first transmission pipeline, which connects the adjacent two levels of the membrane separation module, connects to the second membrane component,
each level of the membrane separation module further includes a permeate condensation component which connects to the membrane component to perform a permeate condensation treatment on a permeate vapor generated by the membrane component,
or each level of the membrane separation module further includes a permeate condensation component, a first membrane component, and a second membrane component, the permeate condensation component in each level of the membrane separation module includes two permeate condensers and two cooling water pipelines, and the membrane separation and purification apparatus further includes a vacuum module and a cold water module, and
the first membrane component connects to one of the permeate condensers, one of the cooling water pipelines introduces water at 5° C. to 15° C. into the permeate condenser connected to the first membrane component, the second membrane component connects to the other permeate condenser, the other cooling water pipeline introduces water at −15° C. to −5° C. into the permeate condenser connected to the second membrane component, and during the membrane separation and purification, a vacuum degree of the permeate condenser, into which water at 5° C. to 15° C. is introduced, in each level of the membrane separation module is controlled to be higher than a vacuum degree of the permeate condenser into which water at −15° C. to −5° C. is introduced.

In some embodiments, a value of the N is 2, 3, or 4,
and/or the membrane separation and purification apparatus further includes a storage module that is used to recover a permeate generated by each level of the membrane separation module, and
the organic azeotrope is selected from any one of alcohols, ketones, aldehydes, ethers, and esters.

In some embodiments, the membrane separation and purification apparatus further includes a cooling module that is used to perform a cooling treatment on the product that has undergone the third heat exchange through the Nth level of the membrane separation module,
or the membrane separation and purification apparatus further includes a cooling module that is used to perform a cooling treatment on the product that has undergone the third heat exchange through the Nth level of the membrane separation module, and a circulating water module that connects to the cooling module.

In some embodiments, the first transmission pipeline connected to the Nth level of the membrane separation module includes a first branch and a second branch,
the membrane separation and purification apparatus further includes a functional heat exchange module,
the first branch connects the Nth level of the membrane separation module and the functional heat exchange module to introduce the product generated by the Nth level of the membrane separation module into the functional heat exchange module to perform a fourth heat exchange,
the second branch connects the functional heat exchange module and the Nth level of the membrane separation module to introduce the product that has undergone the fourth heat exchange into the Nth level of the membrane separation module to perform the third heat exchange,
the functional heat exchange module includes any one of a distillation module and a molecular sieve adsorption module,
the distillation module includes a second heat exchanger, a distillation column, a reboiler, a third condenser, a seventh transmission pipeline, an eighth transmission pipeline, a ninth transmission pipeline, a tenth transmission pipeline, and an eleventh transmission pipeline,
a discharge end of the first branch connects to the reboiler, and a feed end of the second branch connects to the reboiler,
the seventh transmission pipeline connects the second heat exchanger and the distillation column to introduce a material that has passed the second heat exchanger into the distillation column,
the eighth transmission pipeline connects the distillation column and the reboiler to introduce a tower bottom liquid generated by the distillation column into the reboiler,
the ninth transmission pipeline connects the reboiler and the distillation column to transfer the tower bottom liquid heated by the reboiler into the distillation column,
the tenth transmission pipeline connects the reboiler and the second heat exchanger to transfer the tower bottom liquid that has been heated by the reboiler into the second heat exchanger to provide heat to the second heat exchanger,
the eleventh transmission pipeline connects the distillation column and the third condenser to transfer a gaseous azeotropic material discharged from a top of the distillation column into the third condenser to perform a condensation treatment,
the membrane separation and purification apparatus further includes a circulating water module that connects to the third condenser,
and/or the membrane separation and purification apparatus further includes a recovery device that connects to the third condenser to collect an azeotrope discharged by the third condenser,
and/or each level of the membrane separation module also connects to the second heat exchanger to introduce the permeate generated by each level of the membrane separation module into the second heat exchanger.

In some embodiments, the molecular sieve adsorption module includes a third heat exchanger, a twelfth transmission pipeline, a second evaporator, a thirteenth transmission pipeline, and at least one adsorption tower component, wherein the third heat exchanger, the twelfth transmission pipeline, the second evaporator, the thirteenth transmission pipeline, and the adsorption tower component are connected sequentially,
the discharge end of the first branch connects to the second evaporator, and the feed end of the second branch connects to the second evaporator, and
the molecular sieve adsorption module further includes a fourteenth transmission pipeline that connects the adsorption tower component and the third heat exchanger to deliver a vapor generated by the adsorption tower component to the third heat exchanger to perform a sixth heat exchange with a material introduced into the third heat exchanger.

In some embodiments, each level of the membrane separation module generates a permeate, and the permeate generated by any level of the membrane separation module or any of the materials is introduced into the distillation module, or
the permeate generated by any level of the membrane separation module or any of the materials is introduced into the molecular sieve adsorption module.

The implementation of the embodiments of the present application yields the following beneficial effects.

### Advantageous Effects of Invention

Compared to the prior art, the membrane separation and purification apparatus according to the present application includes a heating module and N levels of a membrane separation module, wherein a first level of the membrane separation module is heated by the heating module to perform a membrane separation and purification treatment, adjacent two levels of the membrane separation module are connected via a first transmission pipeline to transfer a product generated by a preceding level of the membrane separation module to a subsequent level of the membrane separation module, a first heat exchange is performed in the subsequent level of the membrane separation module to supply heat to the subsequent level of the membrane separation module, and the product that has undergone the heat exchange in the subsequent level of the membrane separation module is returned to the preceding level of the membrane separation module through a second transmission pipeline to perform a second heat exchange. The membrane separation and purification method based on the membrane separation and purification apparatus not only achieves a cascade of each level of the membrane separation module in the membrane separation and purification apparatus but also ensures a thermal coupling supply of each level of the membrane separation module, and implements a thermal coupling during a membrane separation, thereby effectively improving a heat utilization efficiency of the membrane separation and purification apparatus and reducing energy consumption.

### BRIEF DESCRIPTION OF DRAWINGS

In order to illustrate the embodiments of the present application or the technical solutions in the prior art more clearly, the following briefly introduces the accompanying drawings that need to be used in the description of the embodiments or the prior art. Obviously, the accompanying drawings in the following description are merely some embodiments of the present application, and for a person skilled in the art, other drawings can also be obtained from these accompanying drawings without creative effort.

In the drawings:

FIG. 1 is a simplified structural diagram of a membrane separation and purification apparatus according to an embodiment of the present application.

FIG. 2 is a simplified structural diagram of a membrane separation module according to the embodiment of the present application.

FIG. 3 is a simplified structural diagram of a cooling module according to the embodiment of the present application.

FIG. 4 is a simplified structural diagram of a membrane separation and purification apparatus according to another embodiment of the present application.

FIG. 5 is a simplified structural diagram of a distillation module according to the embodiment of the present application.

FIG. 6 is a simplified structural diagram of a molecular sieve adsorption module according to the embodiment of the present application.

### DESCRIPTION OF EMBODIMENTS

The technical solutions in the embodiments of the present application will be clearly and completely described below with reference to the accompanying drawings in the embodiments of the present application. Obviously, the embodiments to be described are merely some, but not all, embodiments of the present application. Based on the embodiments of the present application, all other embodiments obtained by a person skilled in the art without creative efforts are within the protection scope of the present application.

### Terminology Definitions and Description

In the present application, the terms “preceding level of membrane separation module” and “subsequent level of membrane separation module” are used. These terms do not imply a hierarchical relationship between two membrane separation modules. Instead, they refer to adjacent two membrane separation modules where the membrane separation module supplying heat is defined as the preceding level, and the membrane separation module receiving heat is defined as the subsequent level.

The technical solution of the present application is described in detail below.

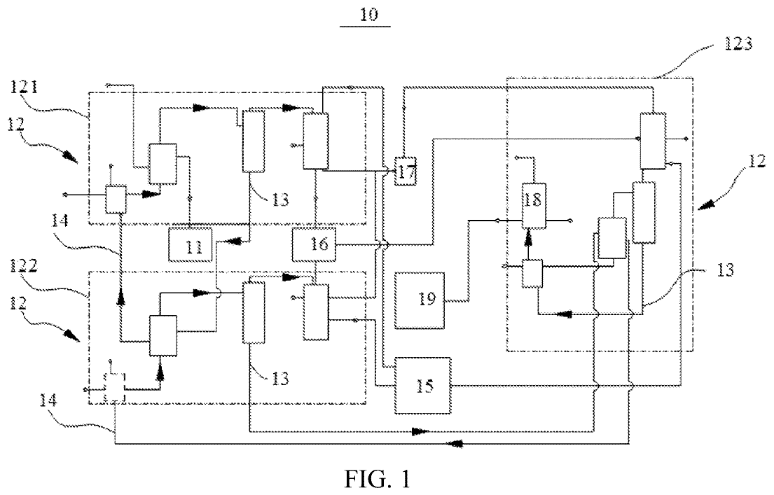

Referring to FIGS. 1 to 3, a membrane separation and purification apparatus 10 according to the embodiment of the present application includes a heating module 11, N levels of a membrane separation module 12, N first transmission pipelines 13, and at least N−1 second transmission pipelines 14, with N being a positive integer not less than 2.

Specifically, each level of the membrane separation module 12 is used to perform a membrane separation and purification treatment on a material to obtain a corresponding product and meanwhile generate a corresponding permeate. A first-level membrane separation module 121 is heated by the heating module 11 to perform the membrane separation and purification treatment. Adjacent two levels of the membrane separation module 12 are connected via a first transmission pipeline 13 and a second transmission pipeline 14, respectively. The first transmission pipeline 13 is used to introduce a product generated by a preceding level of the membrane separation module 12 into a subsequent level of the membrane separation module 12 to perform a first heat exchange with the material introduced into the subsequent level of the membrane separation module 12, thereby supplying heat to the subsequent level of the membrane separation module 12. The second transmission pipeline 14 is used to output the product that has undergone the first heat exchange into the preceding level of the membrane separation module 12 to perform a second heat exchange with the material introduced into the preceding level of the membrane separation module 12, so that the product generated by the preceding level of the membrane separation module 12 undergoes two heat exchanges and the temperature of the product generated by the preceding level of the membrane separation module 12 reaches a temperature suitable for collection. An Nth level of the membrane separation module 12 introduces a product generated by the Nth level of the membrane separation module 12 into the Nth level of the membrane separation module 12 via one first transmission pipeline 13 to perform a third heat exchange with a material introduced into the Nth level of the membrane separation module 12.

When using the membrane separation and purification apparatus 10 for the membrane separation and purification treatment, at least the following steps are included. A material is introduced into each level of the membrane separation module 12, the first-level membrane separation module 121 is heated with the heating module 11 to perform the membrane separation and purification treatment, and a flow rate of a material entering a preceding level of the membrane separation module 12 is controlled to be greater than a flow rate of a material entering a subsequent level of the membrane separation module 12 between adjacent two levels of the membrane separation module 12, wherein each introduced material contains organic azeotropes.

In the membrane separation and purification apparatus 10 according to the present embodiment, the heat transmission and heat supply among a plurality of levels of the membrane separation modules 12 enable a cascade and heat coupling of a plurality of levels of the membrane separation module 12, making full use of the heat carried by the product generated by each level of the membrane separation module 12, and thereby effectively improving a heat utilization efficiency of the membrane separation and purification apparatus 10 and simultaneously reducing the energy consumption of the membrane separation and purification apparatus 10. Therefore, the membrane separation and purification method based on the membrane separation and purification apparatus 10 only requires the heating module 11 to heat the first-level membrane separation module 121 to supply heat for the membrane separation treatment of other levels of the membrane separation module 12 through heat coupling, thereby effectively improving the effective utilization of heat in the membrane separation and purification and reducing energy consumption.

For the present embodiment, as long as a latent heat value of the material introduced into the preceding level of the membrane separation module 12 is greater than a latent heat value of the material introduced into the subsequent level of the membrane separation module 12 and the heating module 11 is controlled to heat the first-level membrane separation module 121, the normal operation of the membrane separation and purification apparatus 10 during the membrane separation and purification can be ensured, and the heat utilization efficiency and energy savings can be effectively improved. In some embodiments, the suitable material to be introduced into each level of the membrane separation module 12 of the membrane separation and purification apparatus 10 of the present embodiment for the membrane separation and purification can be the same material or different materials. In some embodiments, the organic azeotropes contained in the introduced material can be alcohols, ketones, aldehydes, esters, etc. In some embodiments, the introduced material can be aqueous ethanol, and when the flow rate of the aqueous ethanol material introduced into the preceding level of the membrane separation module 12 is greater than the flow rate of the aqueous ethanol material introduced into the subsequent level of the membrane separation module 12, effective utilization of heat in the membrane separation and purification can be achieved. In some embodiments, the introduced material is an aqueous ethanol material, and the water content of the material introduced into each level of the membrane separation module 12 is taken as x, wherein 0<x≤35%, with the water content unit being volume content, mass content, or weight content, depending on the material properties.

In some embodiments, the heating module 11 introduces vapor, and the heating module 11 introduces vapor into a first evaporator 1203 of the first-level membrane separation module 121, allowing for effective heat exchange with the material introduced into the first-level membrane separation module 121 and improving the vaporization efficiency of the material. This heating design can implement heat transmission and heat exchange with the entire membrane separation and purification apparatus 10 by merely supplying heat to the first-level membrane separation module 121 with the heating module 11, thus supplying heat to the entire membrane separation and purification apparatus 10.

In some embodiments, N is 2, meaning that the membrane separation and purification apparatus 10 includes the first-level membrane separation module 121 and a second-level membrane separation module 122. The first-level membrane separation module 121 is heated by the heating module 11 to vaporize the material introduced into the first-level membrane separation module 121, thus performing the membrane separation and purification treatment on the material introduced into the first-level membrane separation module 121 in the first-level membrane separation module 121. The product obtained from the membrane separation and purification treatment in the first-level membrane separation module 121 is introduced through the first transmission pipeline 13 into the second-level membrane separation module 122, supplying heat (first heat exchange) to the second-level membrane separation module 122. This heating causes the material introduced into the second-level membrane separation module 122 to be heated and vaporized, and thus performing the membrane separation and purification treatment on the material introduced into the second-level membrane separation module 122 in the second-level membrane separation module 122. The product from the first-level membrane separation module 121 supplies heat to the second-level membrane separation module 122 and is then transferred through one second transmission pipeline 14 to the first-level membrane separation module 121 for the second heat exchange with the material introduced into the first-level membrane separation module 121, reducing the temperature of the product obtained from the first-level membrane separation module 121 to a suitable temperature for collection. The material entering the first-level membrane separation module 121 has its temperature increased after the second heat exchange and is supplied with heat by the heating module 11 to achieve vaporization. Of course, N is not limited to 2; N can also be 3, 4, 5, or other integers.

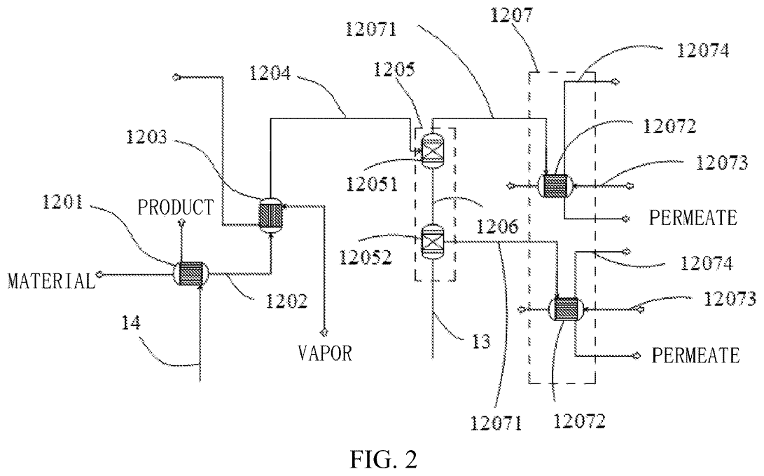

Referring to FIGS. 1 and 2, in some embodiments, each level of the membrane separation module 12 includes a first heat exchanger 1201, a third transmission pipeline 1202, a first evaporator 1203, a fourth transmission pipeline 1204, and at least one membrane component 1205, and the first heat exchanger 1201, the third transmission pipeline 1202, the first evaporator 1203, the fourth transmission pipeline 1204, and the membrane component 1205 are sequentially connected. The heating module 11 supplies heat to the first evaporator 1203 of the first-level membrane separation module 121. Between adjacent two levels of the membrane separation modules 12, the feed end of the first transmission pipeline 13 connects to the membrane component 1205 of the preceding level of the membrane separation module 12, and the discharge end of the first transmission pipeline 13 connects to the first evaporator 1203 of the subsequent level of the membrane separation module 12. The feed end of the second transmission pipeline 14 connects to the first evaporator 1203 of the subsequent level of the membrane separation module 12, and the discharge end of the second transmission pipeline 14 connects to the first heat exchanger 1201 of the preceding level of the membrane separation module 12. This connection arrangement ensures that the product generated by the preceding level of the membrane separation module 12 undergoes the first heat exchange with the material introduced into the subsequent level of the membrane separation module 12, then returns to the current level of the membrane separation module 12 to undergo the second heat exchange with the material introduced into the current level of the membrane separation module 12, and thus, the temperature of the product generated by the current level of the membrane separation module 12 reaches a suitable temperature for collection and the subsequent level of the membrane separation module 12 acquires heat, allowing for the membrane separation and purification treatment. In the present embodiment, the membrane component 1205 is a molecular sieve membrane. Using the molecular sieve membrane as the membrane component 1205 not only reduces apparatus installation space but also allows for a static separation and purification process, avoiding the need to switch towers and achieving a high recovery rate. For example, when the material of separation and purification is aqueous ethanol, the recovery rate of ethanol after passing the membrane component 1205 can reach 99.9% or greater.

In some embodiments, each level of the membrane separation module 12 further includes a fifth transmission pipeline 1206, a first membrane component 12051 and a second membrane component 12052. The fourth transmission pipeline 1204 connects the first evaporator 1203 and the first membrane component 12051 to transfer the material vaporized by the first evaporator 1203 to the first membrane component 12051 for membrane separation and purification. A primary product and a first permeate vapor are obtained from the membrane separation and purification treatment of the first membrane component 12051. The fifth transmission pipeline 1206 connects the first membrane component 12051 and the second membrane component 12052 to transfer the primary product obtained from the membrane separation and purification treatment of the first membrane component 12051 to the second membrane component 12052 for further membrane separation and purification, obtaining the product and a second permeate vapor. The feed end of the first transmission pipeline 13 of adjacent two levels of the membrane separation modules 12 connects to the second membrane component 12052 of the preceding level of the membrane separation module 12 to transfer the product obtained from the preceding level of the membrane separation module 12 to the subsequent level of the membrane separation module 12. By arranging the first membrane component 12051 and the second membrane component 12052, the membrane separation and purification effect can be further improved, thereby facilitating the acquisition of products with higher purity and further improving the heat utilization efficiency.

In some embodiments, each level of the membrane separation module 12 further includes a permeate condensation component 1207 which connects to the membrane component 1205 to perform a permeate condensation treatment on a permeate vapor generated by the membrane component 1205. The permeate condensation component 1207 is arranged to facilitate the collection of the permeate vapor, thereby preventing the permeate vapor from being directly discharged into the surrounding environment and causing adverse effects on the surrounding environment.

In some embodiments, the membrane separation and purification apparatus 10 further includes a vacuum module 15 and a cold water module 16, and the permeation condensation component 1207 includes a sixth transmission pipeline 12071, a permeate condenser 12072, a cooling water pipeline 12073, and a vacuum pipeline 12074. The sixth transmission pipeline 12071 connects the membrane component 1205 and the permeate condenser 12072, so that the permeate vapor generated by the membrane component 1205 can be introduced into the permeate condenser 12072. The cooling water pipeline 12073 connects the cold water module 16 and the permeate condenser 12072, so that water at 15° C. or below can be introduced into the permeate condenser 12072, and the permeate vapor can be condensed by using low-temperature water. The vacuum pipeline 12074 connects the permeate condenser 12072 and the vacuum module 15, so as to create a vacuum condition for the permeate condenser 12072, and by creating the vacuum condition, the effect of permeate condensation can be effectively improved.

In some embodiments, during the membrane separation treatment in the first membrane component 12051, the first permeate vapor is generated, and during the membrane separation treatment in the second membrane component 12052, the second permeate vapor is generated. The first permeate vapor and the second permeate vapor have different compositions and temperatures. Thus, the permeation condensation component 1207 of each level of the membrane separation module 12 includes two permeate condensers 12072 and two cooling water pipelines 12073. The first membrane component 12051 connects to one of the permeate condensers 12072, and one of the cooling water pipelines 12073 is used to introduce water at 5° C. to 15° C. into the permeate condenser 12072 connected to the first membrane component 12051 to aid in condensation. The second membrane component 12052 connects to the other permeate condenser 12072, and the other cooling water pipeline 12073 is used to introduce water at −15° C. to −5° C. into the permeate condenser 12072 connected to the second membrane component 12052 to aid in condensation. The design of the permeate condensation component 1207 in this manner facilitates the condensation treatment of permeate vapors in different states. In some embodiments, each level of the membrane separation module 12 includes two vacuum pipelines 12074. One of the vacuum pipelines 12074 connects to the permeate condenser 12072 which connects to the first membrane component 12051, and the other of the vacuum pipelines 12074 connects to the permeate condenser 12072 which connects to the second membrane component 12052. This arrangement allows for the adjustment of the vacuum conditions of each of the permeate condensers 12072. As for the vacuum conditions of each of the permeate condensers 12072, during the membrane separation and purification treatment, the vacuum level of the permeate condenser 12072, which receives water at 5° C. to 15° C., is controlled to be higher than the vacuum level of the permeate condenser 12072, which receives water at −15° C. to −5° C., in each level of the membrane separation module 12, thus improving the permeate condensation effect. The specific vacuum level can be adjusted based on the purity of the membrane separation and purification, and is not elaborated here.

Refer to FIGS. 1 and 2, in some embodiments, the membrane separation and purification apparatus 10 further includes a storage module 17. By arranging the storage module 17, permeate generated by each level of the membrane separation module 12 can be effectively recovered. In some embodiments, the permeate vapor generated by the first membrane component 12051 is directly discharged into the storage module 17 after being condensed by the permeate condensation component 1207. Similarly, the permeate vapor generated by the second membrane component 12052 is also directly discharged into the storage module 17 after being condensed by the permeate condensation component 1207. In some embodiments, the storage module 17 is a liquid storage tank.

Refer to FIGS. 1, 2, and 3, in some embodiments, the membrane separation and purification apparatus 10 further includes a cooling module 18 that is used to perform a cooling treatment on the product that has undergone the third heat exchange through the Nth level of the membrane separation module 12, so that the temperature of the product generated by the Nth level of the membrane separation module 12 reaches a temperature suitable for collection.

In some embodiments, the cooling module 18 includes a first condenser 181 and a second condenser 182. The first condenser 181 is used to perform a first cooling treatment on the product that has undergone the third heat exchange, while the second condenser 182 is used to perform a second cooling treatment on the product that has undergone the first cooling treatment. After the two steps of cooling, the product in a gas-liquid mixed state after the third heat exchange can be converted into a high-temperature liquid product, which is then converted into a liquid product with a temperature suitable for collection.

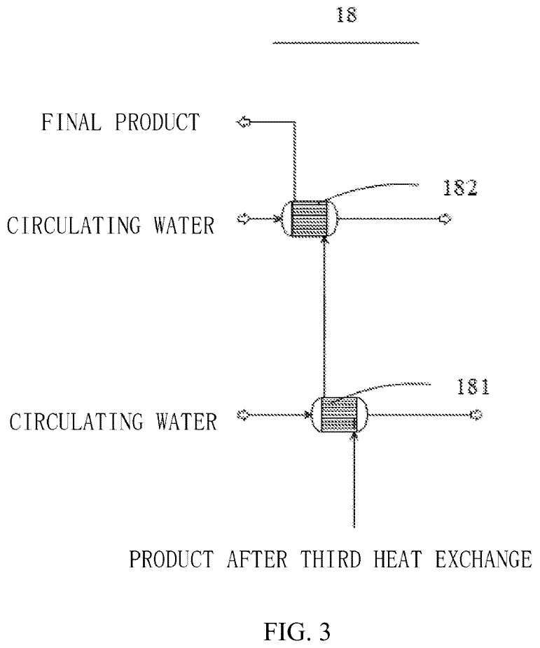

Refer to FIGS. 1 and 3, in some embodiments, the membrane separation and purification apparatus 10 further includes a circulating water module 19, which connects to the cooling module 18 to implement the cooling of the product in the gas-liquid mixed state after the third heat exchange. In some embodiments, the circulating water module 19 connects respectively to the first condenser 181 and the second condenser 182 to implement the cooling respectively.

In the above-described embodiments, the membrane separation and purification apparatus 10, which includes a plurality of levels of the membrane separation module 12, only needs to supply heat to the first-level membrane separation module 121 to achieve coupled heat utilization. Compared to conventional azeotropic distillation, this approach offers advantages such as simplified operation, high product quality, low energy consumption, small footprint, and easy expansion. Compared with molecular sieve adsorption, this approach can achieve continuous operation, does not require frequent switching of heating and regeneration, has low energy consumption, and has the characteristics such as smaller footprint and easy expansion.

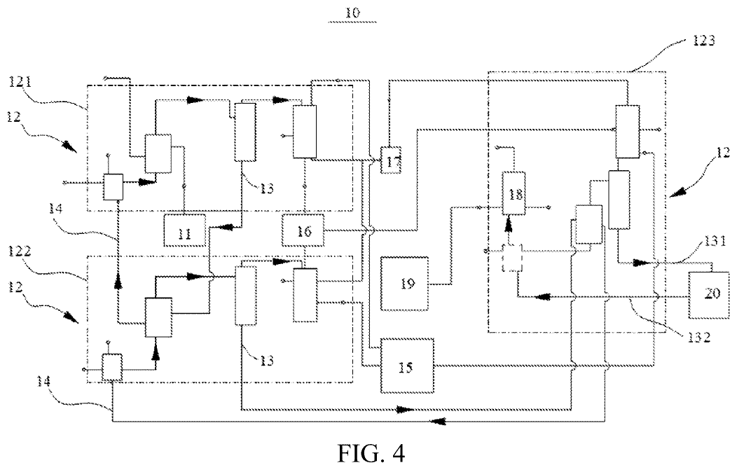

Refer to FIG. 4, in some embodiments, the membrane separation and purification apparatus 10 further includes a functional heat exchange module 20, which utilizes the product generated by the Nth level of the membrane separation module 12 for heat exchange to heat the functional heat exchange module 20, thereby further enhancing the effective utilization of thermal energy carried by the product generated by the Nth level of the membrane separation module 12 among the N levels of the membrane separation module 12.

In some embodiments, the first transmission pipeline 13 connected to the Nth level of the membrane separation module 12 includes a first branch 131 and a second branch 132. The first branch 131 connects the Nth level of the membrane separation module 12 and the functional heat exchange module 20, thereby allowing the product generated by the Nth level of the membrane separation module 12 to be introduced into the functional heat exchange module 20 to perform a fourth heat exchange in the functional heat exchange module 20. The second branch 132 connects the functional heat exchange module 20 and the Nth level of the membrane separation module 12, allowing the product that has undergone the fourth heat exchange to be introduced into the Nth level of membrane separation module 12 to perform the third heat exchange with the material introduced into the Nth level of membrane separation module 12. This structural design not only improves the effective utilization of thermal energy carried by the product generated by the membrane separation module 12 but also makes the structure of the membrane separation and purification apparatus 10 more compact, effectively improving the utilization rate of the installation space of the membrane separation and purification apparatus 10. In some embodiments, the functional heat exchange module 20 can be a distillation module 21 or a molecular sieve adsorption module 22, thereby fully utilizing the thermal energy generated by the membrane separation module 12 for distillation or molecular adsorption treatment.

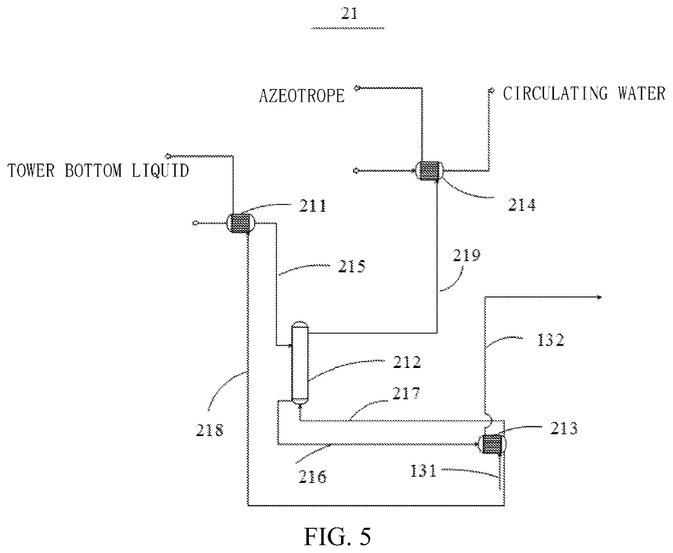

Refer to FIGS. 4, 5, and 6, when the functional heat exchange module 20 is the distillation module 21, the distillation module 21 includes a second heat exchanger 211, a distillation column 212, a reboiler 213, a third condenser 214, a seventh transmission pipeline 215, an eighth transmission pipeline 216, a ninth transmission pipeline 217, a tenth transmission pipeline 218, and an eleventh transmission pipeline 219. The seventh transmission pipeline 215 connects the second heat exchanger 211 and the distillation column 212 to introduce the material that has gone through the second heat exchanger 211 into the distillation column 212. The eighth transmission pipeline 216 connects the distillation column 212 and the reboiler 213 to introduce a tower bottom liquid generated by the distillation column 212 into the reboiler 213 for heating, thus performing the fourth heat exchange with the product transferred from the Nth level of the membrane separation module 12. The ninth transmission pipeline 217 connects the reboiler 213 and the distillation column 212 to return the tower bottom liquid heated by the reboiler 213 to the distillation column 212, replenishing the liquid of the distillation column 212 and performing a secondary distillation. The tenth transmission pipeline 218 connects the reboiler 213 and the second heat exchanger 211 to introduce the tower bottom liquid heated by the reboiler 213 into the second heat exchanger 211, providing a heat source for the second heat exchanger 211 to perform a fifth heat exchange with the material introduced into the distillation module 21 via the second heat exchanger 211. The tower bottom liquid that has undergone the fifth heat exchange can be discharged directly. The eleventh transmission pipeline 219 connects the distillation column 212 and the third condenser 214 to transfer the gaseous azeotropic material discharged from the top of the distillation column 212 into the third condenser 214 for condensation, resulting in azeotropes. In some embodiments, the membrane separation and purification apparatus 10 further includes a recovery device (not shown) for recovering the azeotropes discharged from the third condenser 214. In some embodiments, the material introduced into the distillation module 21 can be any permeate generated by the Nth level of the membrane separation module 12. Introducing any permeate generated by the Nth level of the membrane separation module 12 into the distillation module 21 can effectively enhance the distillation treatment of the permeate, so that the permeate which has undergone the distillation treatment can be directly discharged and the discharge meets the direct discharge standard (GB8978-1996). Or other materials suitable for distillation can also be introduced into the distillation module 21, such as the same material as that in the membrane separation module 12, or a material different from that in the membrane separation module 12. In some embodiments, the circulating water module 19 also connects to the third condenser 214 to assist in cooling the gaseous azeotrope generated by the distillation tower 212, so that the gaseous azeotrope can be effectively cooled and converted into a liquid azeotrope for easy collection.

In the present embodiment, by combining the membrane separation module 12 with distillation, thermal coupling with distillation is achieved on the basis of achieving thermal coupling of the plurality of levels of the membrane separation module 12, so that the molecular sieve membrane and distillation can be used in combination, which is beneficial to improving heat utilization efficiency and saving energy consumption. When aqueous ethanol is used as the raw material for separation and purification, the water content in the ethanol vapor at the top of the distillation tower 212 can reach a relatively high value, and the distillation process does not need to reach azeotropy, which is beneficial to reducing the energy consumption of the distillation tower 212, so that the distillation conditions of the distillation tower 212 are reduced. In addition, since the single-pass recovery rate of the membrane separation module 12 reaches 99.5% or more, the energy consumption of the entire apparatus in the separation and purification of aqueous ethanol is further reduced. In the present embodiment, the membrane separation and purification apparatus 10 adopts a combination of the membrane separation module 12 and the distillation module 20. When dehydrating and purifying ethanol, compared with the combination of molecular sieve adsorption and distillation, for every ton of water-containing ethanol material separated and purified, the heating module 11 can save 180 kg or more of vapor introduction, the consumption of circulating water can be saved by 10 tons or more, and 5 kWh or more of electricity can be saved. Therefore, it not only saves energy consumption but also reduces the cost of separation and purification.

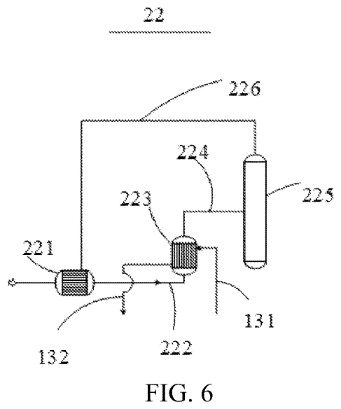

Referring to FIGS. 4 and 6, when the functional heat exchange module 20 is the molecular sieve adsorption module 22, the molecular sieve adsorption module 22 includes a third heat exchanger 221, a twelfth transmission pipeline 222, a second evaporator 223, a thirteenth transmission pipeline 224, and at least one adsorption tower component 225. The third heat exchanger 221, the twelfth transmission pipeline 222, the second evaporator 223, the thirteenth transmission pipeline 224, and the adsorption tower component 225 are connected sequentially. The feed end of the first branch 131 connects to the membrane component 1205 of the Nth level of the membrane separation module 12, and the discharge end of the first branch 131 connects to the second evaporator 223. The feed end of the second branch 132 connects to the second evaporator 223, and the discharge end of the second branch 132 connects to the first heat exchanger 1201 of the Nth level of the membrane separation module 12. In some embodiments, the molecular sieve adsorption module 22 further includes a fourteenth transmission pipeline 226 that connects the adsorption tower component 225 and the third heat exchanger 221 to deliver a vapor generated by the adsorption tower component 225 to the third heat exchanger 221 to perform a sixth heat exchange with a material introduced into the third heat exchanger 221. The material introduced into the molecular sieve adsorption module 22 may be the same as the material used in the membrane separation module 12 or a material different from the material used in the membrane separation module 12, and of course, may also be any permeate generated by the Nth level of the membrane separation module 12. This structural design can further improve the heat utilization efficiency of the molecular sieve adsorption module 22, reducing external heat supply and thereby lowering energy consumption.

Referring to FIGS. 1 to 3, the basic working principle of the membrane separation and purification apparatus 10 according to the present embodiment is described as follows with a structure including three levels of the membrane separation module 12.

### (1)<Basic Working Process of First-Level Membrane Separation Module 121>

A first material is introduced into the first-level membrane separation module 121. There is no heat exchange process when a first batch of the first material passes through the first heat exchanger 1201. The first material is transferred to the first evaporator 1203. At the same time, the heating module 11 introduces vapor into the first evaporator 1203. The first material undergoes a first heat exchange with the vapor when passing through the first evaporator 1203. The first material is vaporized into a gaseous material and is transferred to the first membrane component 12051 for a first membrane separation treatment. The gaseous first material after the first membrane separation treatment is divided into a first primary product and a first permeate vapor. The first primary product is transferred to the second membrane component 12052 for a second membrane separation treatment. At the same time, the first permeate vapor is transferred to the permeate condenser 12072 connected to the first membrane component 12051 for a first permeate condensation treatment and produces a corresponding permeate. When the first permeate vapor is undergoing the first permeate condensation, the cold water module 16 introduces water at 5° C. to 15° C. into the permeate condenser 12072 connected to the first membrane component 12051. The vacuum module 15 works so that the permeate condenser 12072 connected to the first membrane component 12051 forms a certain degree of vacuum to assist the permeate condensation. The first primary product after the second membrane separation treatment is divided into a first product and a second permeate vapor, wherein the second permeate vapor is transferred to the permeate condenser 12072 connected to the second membrane component 12052 for the second permeate condensation treatment and produces a corresponding permeate. When the second permeate vapor is undergoing the second permeate condensation, the cold water module 16 introduces water at −15° C. to −5° C. into the permeate condenser 12072 connected to the second membrane component 12052 to form a certain degree of vacuum, and the vacuum degree is lower than the vacuum degree of the permeate condenser 12072, into which water at 5° C. to 15° C. is introduced, to assist the permeate condensation. The first product enters the first evaporator 1203 of the second-level membrane separation module 122 via the first transmission pipeline 13, and performs a heat exchange in the first evaporator 1203 of the second-level membrane separation module 122.

### (2)<Basic Working Process of Second-Level Membrane Separation Module 122>

A second material is introduced into the second-level membrane separation module 122. There is no heat exchange process when a first batch of the second material passes through the first heat exchanger 1201. The second material is transferred to the first evaporator 1203, and undergoes a first heat exchange with the first product transferred to the first evaporator 1203 by the first transmission pipeline 13 connected to the first-level membrane separation module 121 and the second-level membrane separation module 122. The second material becomes gaseous and is then transferred to the first membrane component 12051 for the first membrane separation treatment. The gaseous second material after the first membrane separation treatment is separated into a second primary product and a third permeate vapor. The second primary product is transferred into the second membrane component 12052 for the second membrane separation treatment. At the same time, the third permeate vapor is transferred into the permeate condenser 12072 connected to the first membrane component 12051 for a second permeate condensation treatment and produces a corresponding permeate. When the third permeate vapor is undergoing the first permeate condensation, the cold water module 16 introduces water at 5° C. to 15° C. into the permeate condenser 12072 connected to the first membrane component 12051, and the vacuum module 15 works to form a certain degree of vacuum in the permeate condenser 12072 connected to the first membrane component 12051 to assist the permeate condensation. The second primary product after the second membrane separation treatment is divided into a second product and a fourth permeate vapor, wherein the fourth permeate vapor is transferred into the permeate condenser 12072 connected to the second membrane component 12052 for a second permeate condensation treatment and produces a corresponding permeate. When the fourth permeate vapor is undergoing the second permeate condensation, the cold water module 16 introduces water at −15° C. to −5° C. into the permeate condenser 12072 connected to the second membrane component 12052, and forms a certain degree of vacuum, and the vacuum degree is lower than the vacuum degree of the permeate condenser 12072, into which water at 5° C. to 15° C. is introduced, to assist the permeate condensation. The second product enters the first evaporator 1203 of the third-level membrane separation module 123 via the first transmission pipeline 13, and performs a heat exchange in the first evaporator 1203 of the third-level membrane separation module 123. At the same time, the first product that has undergone the first heat exchange is introduced into the first heat exchanger 1201 of the first-level membrane separation module 121 via the second transmission pipeline 14 which connects the first-level membrane separation module 121 and the second-level membrane separation module 122, and undergoes a second heat exchange with the first material subsequently flowing through the first heat exchanger 1201. At this point, the first product separated by the first-level membrane separation module 121 has undergone two heat exchanges, and the temperature of the first product is lowered to a temperature suitable for collection.

### (3)<Basic Working Process of Third-Level Membrane Separation Module 123>

A third material is introduced into the third-level membrane separation module 123. There is no heat exchange when a first batch of the third material passes through the first heat exchanger 1201. The third material is transferred to the first evaporator 1203, and undergoes a first heat exchange with the second product transferred to the first evaporator 1203 via the first transmission pipeline 13 connected to the second-level membrane separation module 122 and the third-level membrane separation module 123. The third material becomes gaseous and is then transferred to the first membrane component 12051 for the first membrane separation treatment. The gaseous third material after the first membrane separation treatment is divided into a third primary product and a fifth permeate vapor. The third primary product is transferred into the second membrane component 12052 for a second membrane separation treatment. At the same time, the fifth permeate vapor is transferred into the permeate condenser 12072 connected to the first membrane component 12051 for a third permeate condensation treatment and produces a corresponding permeate. When the fifth permeate vapor is undergoing the third permeate condensation, the cold water module 16 introduces water at 5° C. to 15° C. into the permeate condenser 12072 connected to the first membrane component 12051, and the vacuum module 15 works to form a certain degree of vacuum in the permeate condenser 12072 connected to the first membrane component 12051 to assist the permeate condensation. The third primary product after the second membrane separation treatment is divided into a third product and a sixth permeate vapor. The sixth permeate vapor is transferred into the permeate condenser 12072 connected to the second membrane component 12052 for the third permeate condensation treatment and produces a corresponding permeate. When the sixth permeate vapor is undergoing the third permeate condensation, the cold water module 16 introduces water at −15° C. to −5° C. into the permeate condenser 12072 connected to the second membrane component 12052, and forms a certain degree of vacuum, and the vacuum degree is lower than the vacuum degree of the permeate condenser 12072, into which water at 5° C. to 15° C. is introduced, to assist the permeate condensation. The third product enters the first heat exchanger 1201 of the third-level membrane separation module 123 via the first transmission pipeline 13, and undergoes a heat exchange (i.e., a third heat exchange) in the first heat exchanger 1201 of the third-level membrane separation module 123. At the same time, the second product that has undergone the first heat exchange is transferred into the first heat exchanger 1201 of the second-level membrane separation module 122 via the second transmission pipeline 14 which connects the second-level membrane separation module 122 and the third-level membrane separation module 123, and undergoes a second heat exchange with the second material that subsequently flows through the first heat exchanger 1201. At this point, the second product separated by the second-level membrane separation module 122 has undergone two heat exchanges, and its temperature is lowered to a temperature suitable for collection.

(4) The first material, the second material, and the third material initially introduced into the membrane separation and purification apparatus 10 do not undergo any heat exchange when flowing through the first heat exchanger 1201 of the respective level of the membrane separation module 12. The first material, the second material and the third material that flow in subsequently undergo a heat exchange in the first heat exchanger 1201 of the respective level of the membrane separation module 12 only after a corresponding product is generated from each level of the membrane separation module 12. The third product generated by the third-level membrane separation module 123 is in a gas-liquid mixed state after undergoing the third heat exchange with the material which flows into the first heat exchanger 1201 of the third-level membrane separation module 123, and is then cooled by the cooling module 18 to be transformed into a high-temperature liquid which is a liquid product with a temperature suitable for collection.

It should be noted that the numerals (1) to (4) do not limit the sequence of operation for the membrane separation and purification apparatus 10.

In order to better illustrate the solution of the present application, further description is given below through embodiments.

### First Embodiment

Referring to FIGS. 2, 3, 4, and 5, a membrane separation and purification method for ethanol adopts the membrane separation and purification apparatus 10. The membrane separation and purification apparatus 10 includes two levels of the membrane separation module 12 and the distillation module 21, with each level of the membrane separation module 12 including the first membrane component 12051, the second membrane component 12052, and two permeate condensers 12072. The method includes the following steps.

An ethanol material with a water content of approximately 5 v/v % (i.e., the first material) is introduced into the first-level membrane separation module 121, with a flow rate of 600 kg/h. Simultaneously, a vapor with a gauge pressure of 0.5 MPaG and a temperature of 160° C. is introduced into the heating module 11. The evaporation pressure in the first evaporator 1203 of the first-level membrane separation module 121 is set to 0.4 MPa, and the evaporation temperature is set to 124.5° C. In the first-level membrane separation module 121, low-temperature water at 5° C. is introduced into the permeate condenser 12072 connected to the first membrane component 12051, and the vacuum degree of the permeate condenser 12072 connected to the first membrane component 12051 is controlled to be 3 kPa. Freezing water at −10° C. is introduced into the permeate condenser 12072 connected to the second membrane component 12052, and the vacuum degree of the permeate condenser 12072 connected to the second membrane component 12052 is controlled to be 0.3 kPa.

At the same time, an ethanol material with a water content of 5 v/v % (i.e., the second material) is introduced into the second-level membrane separation module 122, with a flow rate of 400 kg/h. The evaporation pressure in the first evaporator 1203 of the second-level membrane separation module 122 is set to 0.2 MPa, and the evaporation temperature is set to 108.2° C. Low-temperature water at 5° C. is introduced into the permeate condenser 12072 connected to the first membrane component 12051, and the vacuum degree of the permeate condenser 12072 connected to the first membrane component 12051 is controlled to be 3 kPa. Freezing water at −10° C. is introduced into the permeate condenser 12072 connected to the second membrane component 12052, and the vacuum degree of the permeate condenser 12072 connected to the second membrane component 12052 is controlled to be 0.3 kPa.

Circulating water is introduced into the membrane separation and purification apparatus 10, with the circulating water flowing through the first condenser 181, the second condenser 182, and the third condenser 214. The permeate generated by the first-level membrane separation module 121 and the permeate generated by the second-level membrane separation module 122 are introduced into the distillation module 21 for distillation.

During the operation of the membrane separation and purification apparatus 10, wastewater generated by the second heat exchanger 211 is collected and tested. The COD of the wastewater is approximately 42.7 mg/L. Meanwhile, the ethanol product obtained from the first-level membrane separation module 121 (i.e., the first product) and the ethanol product obtained from the second-level membrane separation module 122 (i.e., the second product) are collected. The purities of the two collected ethanol products are tested, and the water contents of the two collected ethanol products are 185 ppm and 176 ppm, respectively. In addition, the azeotrope discharged from the third condenser 214 is recycled.

Utilizing the method of the present embodiment to separate and purify 10 tons of ethanol material with a water content of 5 v/v % can achieve a 40% reduction in energy consumption. The amount of vapor introduced into the first-level membrane separation module 121 is reduced by 0.21 tons per ton of product, effectively improving the heat utilization efficiency of the ethanol membrane separation and purification process, with the generated wastewater meeting the direct discharge standard of ≤50 mg/L (GB8978-1996).

### Second Embodiment

Referring to FIGS. 2, 3, 4, and 5, a membrane separation and purification method for isopropanol adopts the membrane separation and purification apparatus 10. The membrane separation and purification apparatus 10 includes two levels of the membrane separation module 12 and the distillation module 21, with each level of the membrane separation module 12 including the first membrane component 12051, the second membrane component 12052, and two permeate condensers 12072. The method includes the following steps.

An isopropanol material with a water content of 5 wt % (i.e., the first material) is introduced into the first-level membrane separation module 121, with a flow rate of 600 kg/h. Simultaneously, a vapor with a gauge pressure of 0.5 MPaG and a temperature of 160° C. is introduced into the heating module 11. The evaporation pressure in the first evaporator 1203 of the first-level membrane separation module 121 is set to 0.4 MPa, and the evaporation temperature is set to 129.6° C. In the first-level membrane separation module 121, low-temperature water at 5° C. is introduced into the permeate condenser 12072 connected to the first membrane component 12051, and the vacuum degree of the permeate condenser 12072 connected to the first membrane component 12051 is controlled to be 3 kPa. Freezing water at −10° C. is introduced into the permeate condenser 12072 connected to the second membrane component 12052, and the vacuum degree of the permeate condenser 12072 connected to the second membrane component 12052 is controlled to be 0.3 kPa.

At the same time, an isopropanol material with a water content of 5 wt % (i.e., the second material) is introduced into the second-level membrane separation module 122, with a flow rate of 400 kg/h. The evaporation pressure in the first evaporator 1203 of the second-level membrane separation module 122 is set to 0.2 MPa, and the evaporation temperature is set to 112.8° C. Low-temperature water at 5° C. is introduced into the permeate condenser 12072 connected to the first membrane component 12051, and the vacuum degree of the permeate condenser 12072 connected to the first membrane component 12051 is controlled to be 3 kPa. Freezing water at −10° C. is introduced into the permeate condenser 12072 connected to the second membrane component 12052, and the vacuum degree of the permeate condenser 12072 connected to the second membrane component 12052 is controlled to be 0.3 kPa.

Circulating water is introduced into the membrane separation and purification apparatus 10, with the circulating water flowing through the first condenser 181, the second condenser 182, and the third condenser 214. The permeate generated by the first-level membrane separation module 121 and the permeate generated by the second-level membrane separation module 122 are introduced into the distillation module 21 for distillation.

During the operation of the membrane separation and purification apparatus 10, wastewater generated by the second heat exchanger 211 is collected and tested. The COD of the wastewater is approximately 39.1 mg/L. Meanwhile, the isopropanol product obtained from the first-level membrane separation module 121 (i.e., the first product) and the isopropanol product obtained from the second-level membrane separation module 122 (i.e., the second product) are collected. The purities of the two collected isopropanol products are tested, and the water contents of the two collected isopropanol products are 100 ppm and 100 ppm, respectively. In addition, the azeotrope discharged from the third condenser 214 is recycled.

Utilizing the method of the present embodiment to separate and purify 5 tons of ethanol material with a water content of 5 wt % can achieve a 40% reduction in energy consumption. The amount of vapor introduced into the first-level membrane separation module 121 is reduced by 0.2 tons per ton of product, effectively improving the heat utilization efficiency of the isopropanol membrane separation and purification process, with the generated wastewater meeting the direct discharge standard of ≤50 mg/L (GB8978-1996).

In summary, when performing membrane separation and purification of azeotropic organic substances such as ethanol and isopropanol, the membrane separation and purification apparatus 10 according to the embodiments of the present application exhibits high heat utilization efficiency, can effectively save energy consumption, and the wastewater generated can meet the direct discharge standard.

### REFERENCE SIGNS LIST

10 Membrane separation and purification apparatus
11 Heating module
12 Membrane separation module
121 First-level membrane separation module
122 Second-level membrane separation module
123 Third-level membrane separation module
1201 First heat exchanger
1202 Third transmission pipeline
1203 First evaporator
1204 Fourth transmission pipeline
1205 Membrane component
12051 First membrane component
12052 Second membrane component
1206 Fifth transmission pipeline
1207 Permeate condensation component
12071 Sixth transmission pipeline
12072 Permeate condenser
12073 Cooling water pipeline
12074 Vacuum pipeline
13 First transmission pipeline
131 First branch
132 Second branch
14 Second transmission pipeline
15 Vacuum module
16 Cold water module
17 Storage module
18 Cooling module
181 First condenser
182 Second condenser
19 Circulating water module
20 Functional heat exchange module
21 Distillation module
211 Second heat exchanger
212 Distillation column
213 Reboiler
214 Third condenser
215 Seventh transmission pipeline
216 Eighth transmission pipeline
217 Ninth transmission pipeline
218 Tenth transmission pipeline
219 Eleventh transmission pipeline
22 Molecular sieve adsorption module
221 Third heat exchanger
222 Twelfth transmission pipeline
223 Second evaporator
224 Thirteenth transmission pipeline
225 Adsorption tower component
226 Fourteenth transmission pipeline

1. A membrane separation and purification apparatus comprising:
a heating module;
N levels of a membrane separation module;
N first transmission pipelines; and
at least N−1 second transmission pipelines,
with N being a positive integer not less than 2, wherein
each level of the membrane separation module is used to perform a membrane separation and purification treatment on a material to obtain a product correspondingly,
a first level of the membrane separation module is heated by the heating module to perform the membrane separation and purification treatment,
adjacent two levels of the membrane separation module are connected respectively via one of the first transmission pipelines and one of the second transmission pipelines, the first transmission pipeline introducing the product generated by a preceding level of the membrane separation module into a subsequent level of the membrane separation module to perform a first heat exchange with a material introduced into the subsequent level of the membrane separation module, the second transmission pipeline outputting the product that has undergone the first heat exchange into the preceding level of the membrane separation module to perform a second heat exchange with a material introduced into the preceding level of the membrane separation module, and
an Nth level of the membrane separation module introduces the product generated by the Nth level of the membrane separation module into the Nth level of the membrane separation module via one of the first transmission pipelines to perform a third heat exchange with a material introduced into the Nth level of the membrane separation module.

2. The membrane separation and purification apparatus according to claim 1, wherein: each level of the membrane separation module includes a first heat exchanger, a third transmission pipeline, a first evaporator, a fourth transmission pipeline, and at least one membrane component, which are connected sequentially,
the heating module provides heat to the first evaporator in the first level of the membrane separation module, and
between the adjacent two levels of the membrane separation module, a feed end of the first transmission pipeline connects to the membrane component in the preceding level of the membrane separation module, and a discharge end of the first transmission pipeline connects to the first evaporator of the subsequent level of the membrane separation module, a feed end of the second transmission pipeline connects to the first evaporator of the subsequent level of the membrane separation module, and a discharge end of the second transmission pipeline connects to the first heat exchanger of a current level of the membrane separation module.

3. The membrane separation and purification apparatus according to claim 2, wherein: each level of the membrane separation module further includes a fifth transmission pipeline, and each level of the membrane separation module includes a first membrane component and a second membrane component,
the fourth transmission pipeline connects the first evaporator and the first membrane component, the fifth transmission pipeline connects the first membrane component and the second membrane component, one end of the first transmission pipeline, which connects the adjacent two levels of the membrane separation module, connects to the second membrane component,
each level of the membrane separation module further includes a permeate condensation component which connects to the membrane component to perform a permeate condensation treatment on a permeate vapor generated by the membrane component,
or each level of the membrane separation module further includes a permeate condensation component, a first membrane component, and a second membrane component, the permeate condensation component in each level of the membrane separation module includes two permeate condensers and two cooling water pipelines, and the membrane separation and purification apparatus further includes a vacuum module and a cold water module, and
the first membrane component connects to one of the permeate condensers, one of the cooling water pipelines introduces water at 5° C. to 15° C. into the permeate condenser connected to the first membrane component, the second membrane component connects to the other permeate condenser, and the other cooling water pipeline introduces water at −15° C. to −5° C. into the permeate condenser connected to the second membrane component.

4. The membrane separation and purification apparatus according to claim 1, wherein: a value of the N is 2, 3, or 4,
and/or the membrane separation and purification apparatus further comprises a storage module that is used to recover a permeate generated by each level of the membrane separation module.

5. The membrane separation and purification apparatus according to claim 1, further comprising: a cooling module that is used to perform a cooling treatment on the product that has undergone the third heat exchange through the Nth level of the membrane separation module,
or the membrane separation and purification apparatus further includes a cooling module that is used to perform a cooling treatment on the product that has undergone the third heat exchange through the Nth level of the membrane separation module, and a circulating water module that connects to the cooling module.

6. The membrane separation and purification apparatus according to claim 1, wherein: the first transmission pipeline connected to the Nth level of the membrane separation module includes a first branch and a second branch,
the membrane separation and purification apparatus further includes a functional heat exchange module,
the first branch connects the Nth level of the membrane separation module and the functional heat exchange module to introduce the product generated by the Nth level of the membrane separation module into the functional heat exchange module to perform a fourth heat exchange,
the second branch connects the functional heat exchange module and the Nth level of the membrane separation module to introduce the product that has undergone the fourth heat exchange into the Nth level of the membrane separation module to perform the third heat exchange,
the functional heat exchange module includes any one of a distillation module and a molecular sieve adsorption module,
the distillation module includes a second heat exchanger, a distillation column, a reboiler, a third condenser, a seventh transmission pipeline, an eighth transmission pipeline, a ninth transmission pipeline, a tenth transmission pipeline, and an eleventh transmission pipeline,
a discharge end of the first branch connects to the reboiler, and a feed end of the second branch connects to the reboiler,
the seventh transmission pipeline connects the second heat exchanger and the distillation column to introduce a material that has passed the second heat exchanger into the distillation column,
the eighth transmission pipeline connects the distillation column and the reboiler to introduce a tower bottom liquid generated by the distillation column into the reboiler,
the ninth transmission pipeline connects the reboiler and the distillation column to transfer the tower bottom liquid heated by the reboiler into the distillation column,
the tenth transmission pipeline connects the reboiler and the second heat exchanger to transfer the tower bottom liquid that has been heated by the reboiler into the second heat exchanger to provide heat to the second heat exchanger,
the eleventh transmission pipeline connects the distillation column and the third condenser to transfer a gaseous azeotropic material discharged from a top of the distillation column into the third condenser to perform a condensation treatment,
the membrane separation and purification apparatus further includes a circulating water module that connects to the third condenser,
and/or the membrane separation and purification apparatus further includes a recovery device that connects to the third condenser to collect an azeotrope discharged by the third condenser,
and/or each level of the membrane separation module also connects to the second heat exchanger to introduce the permeate generated by each level of the membrane separation module into the second heat exchanger.

7. The membrane separation and purification apparatus according to claim 6, wherein: the molecular sieve adsorption module includes a third heat exchanger, a twelfth transmission pipeline, a second evaporator, a thirteenth transmission pipeline, and at least one adsorption tower component, which are connected sequentially,
the discharge end of the first branch connects to the second evaporator, and the feed end of the second branch connects to the second evaporator, and
the molecular sieve adsorption module further includes a fourteenth transmission pipeline that connects the adsorption tower component and the third heat exchanger to deliver a vapor generated by the adsorption tower component to the third heat exchanger to perform a sixth heat exchange with a material introduced into the third heat exchanger.

8. A membrane separation and purification method comprising:
introducing a material into each level of a membrane separation module and heating a first level of the membrane separation module through a heating module to perform a membrane separation and purification treatment; and
controlling adjacent two levels of the membrane separation module so that a flow rate of a material entering a preceding level of the membrane separation module is greater than a flow rate of a material entering a subsequent level of the membrane separation module and a latent heat value of a material entering the preceding level of the membrane separation module is not less than a latent heat value of a material entering the subsequent level of the membrane separation module, wherein
the membrane separation and purification method utilizes a membrane separation and purification apparatus to perform membrane separation and purification,
the membrane separation and purification apparatus includes: the heating module, N levels of the membrane separation module, N first transmission pipelines, and at least N−1 second transmission pipelines, with N being a positive integer not less than 2,
each level of the membrane separation module is used to perform the membrane separation and purification treatment on a material to obtain a product correspondingly,
the first level of the membrane separation module is heated by the heating module to perform the membrane separation and purification treatment,
adjacent two levels of the membrane separation module are connected respectively via one of the first transmission pipelines and one of the second transmission pipelines, the first transmission pipeline introducing a product generated by a preceding level of the membrane separation module into a subsequent level of the membrane separation module to perform a first heat exchange with a material introduced into the subsequent level of the membrane separation module, the second transmission pipeline outputting the product that has undergone the first heat exchange into the preceding level of the membrane separation module to perform a second heat exchange with a material introduced into the preceding level of the membrane separation module,
an Nth level of the membrane separation module introduces a product generated by the Nth level of the membrane separation module into the Nth level of the membrane separation module via one of the first transmission pipelines to perform a third heat exchange with a material introduced into the Nth level of the membrane separation module, and
each of the materials contains an organic azeotrope.

9. The membrane separation and purification method according to claim 8, wherein: each level of the membrane separation module includes a first heat exchanger, a third transmission pipeline, a first evaporator, a fourth transmission pipeline, and at least one membrane component, which are connected sequentially,
the heating module provides heat to the first evaporator in the first level of the membrane separation module, and
between the adjacent two levels of the membrane separation module, a feed end of the first transmission pipeline connects to the membrane component in the preceding level of the membrane separation module, and a discharge end of the first transmission pipeline connects to the first evaporator of the subsequent level of the membrane separation module, a feed end of the second transmission pipeline connects to the first evaporator of the subsequent level of the membrane separation module, and a discharge end of the second transmission pipeline connects to the first heat exchanger of a current level of the membrane separation module.

10. The membrane separation and purification method according to claim 9, wherein: each level of the membrane separation module further includes a fifth transmission pipeline, and each level of the membrane separation module includes a first membrane component and a second membrane component,
the fourth transmission pipeline connects the first evaporator and the first membrane component, the fifth transmission pipeline connects the first membrane component and the second membrane component, one end of the first transmission pipeline, which connects the adjacent two levels of the membrane separation module, connects to the second membrane component,
each level of the membrane separation module further includes a permeate condensation component which connects to the membrane component to perform a permeate condensation treatment on a permeate vapor generated by the membrane component,
or each level of the membrane separation module further includes a permeate condensation component, a first membrane component, and a second membrane component, the permeate condensation component in each level of the membrane separation module includes two permeate condensers and two cooling water pipelines, and the membrane separation and purification apparatus further includes a vacuum module and a cold water module, and
the first membrane component connects to one of the permeate condensers, one of the cooling water pipelines introduces water at 5° C. to 15° C. into the permeate condenser connected to the first membrane component, the second membrane component connects to another permeate condenser, another cooling water pipeline introduces water at −15° C. to −5° C. into the permeate condenser connected to the second membrane component, and during the membrane separation and purification, a vacuum degree of the permeate condenser, into which water at 5° C. to 15° C. is introduced, in each level of the membrane separation module is controlled to be higher than a vacuum degree of the permeate condenser into which water at −15° C. to −5° C. is introduced.

11. The membrane separation and purification method according to claim 8, wherein: a value of the N is 2, 3, or 4,
and/or the membrane separation and purification apparatus further includes a storage module that is used to recover a permeate generated by each level of the membrane separation module, and
the organic azeotrope is selected from any one of alcohols, ketones, aldehydes, ethers, and esters.

12. The membrane separation and purification method according to claim 8, wherein: the membrane separation and purification apparatus further includes a cooling module that is used to perform a cooling treatment on the product that has undergone the third heat exchange through the Nth level of the membrane separation module,
or the membrane separation and purification apparatus further includes a cooling module that is used to perform a cooling treatment on the product that has undergone the third heat exchange through the Nth level of the membrane separation module, and a circulating water module that connects to the cooling module.

13. The membrane separation and purification method according to claim 8, wherein: the first transmission pipeline connected to the Nth level of the membrane separation module includes a first branch and a second branch,
the membrane separation and purification apparatus further includes a functional heat exchange module,
the first branch connects the Nth level of the membrane separation module and the functional heat exchange module to introduce the product generated by the Nth level of the membrane separation module into the functional heat exchange module to perform a fourth heat exchange,
the second branch connects the functional heat exchange module and the Nth level of the membrane separation module to introduce the product that has undergone the fourth heat exchange into the Nth level of the membrane separation module to perform the third heat exchange,
the functional heat exchange module includes any one of a distillation module and a molecular sieve adsorption module,
the distillation module includes a second heat exchanger, a distillation column, a reboiler, a third condenser, a seventh transmission pipeline, an eighth transmission pipeline, a ninth transmission pipeline, a tenth transmission pipeline, and an eleventh transmission pipeline,
a discharge end of the first branch connects to the reboiler, and a feed end of the second branch connects to the reboiler,
the seventh transmission pipeline connects the second heat exchanger and the distillation column to introduce a material that has passed the second heat exchanger into the distillation column,
the eighth transmission pipeline connects the distillation column and the reboiler to introduce a tower bottom liquid generated by the distillation column into the reboiler,
the ninth transmission pipeline connects the reboiler and the distillation column to transfer the tower bottom liquid heated by the reboiler into the distillation column,
the tenth transmission pipeline connects the reboiler and the second heat exchanger to transfer the tower bottom liquid that has been heated by the reboiler into the second heat exchanger to provide heat to the second heat exchanger,
the eleventh transmission pipeline connects the distillation column and the third condenser to transfer a gaseous azeotropic material discharged from a top of the distillation column into the third condenser to perform a condensation treatment,
the membrane separation and purification apparatus further includes a circulating water module that connects to the third condenser,
and/or the membrane separation and purification apparatus further includes a recovery device that connects to the third condenser to collect an azeotrope discharged by the third condenser,
and/or each level of the membrane separation module also connects to the second heat exchanger to introduce the permeate generated by each level of the membrane separation module into the second heat exchanger.

14. The membrane separation and purification method according to claim 13, wherein: the molecular sieve adsorption module includes a third heat exchanger, a twelfth transmission pipeline, a second evaporator, a thirteenth transmission pipeline, and at least one adsorption tower component, and the third heat exchanger, the twelfth transmission pipeline, the second evaporator, the thirteenth transmission pipeline, and the adsorption tower component are connected sequentially,
the discharge end of the first branch connects to the second evaporator, and the feed end of the second branch connects to the second evaporator, and
the molecular sieve adsorption module further includes a fourteenth transmission pipeline that connects the adsorption tower component and the third heat exchanger to deliver a vapor generated by the adsorption tower component to the third heat exchanger to perform a sixth heat exchange with a material introduced into the third heat exchanger.

15. The membrane separation and purification method according to claim 13, wherein: each level of the membrane separation module generates a permeate, and the permeate generated by any level of the membrane separation module or any of the materials is introduced into the distillation module, or
the permeate generated by any level of the membrane separation module or any of the materials is introduced into the molecular sieve adsorption module.
